(19) United States
(12) Patent Application Publication
(10) Pub. No.: US 2012/0215263 A1
(43) Pub. Date: Aug. 23, 2012

(54) EXTENSIBLE PEDICLE SCREW COUPLING DEVICE

(76) Inventor: Choon Sung Lee, Seoul (KR)

(21) Appl. No.: 13/188,805

(22) Filed: Jul. 22, 2011

Related U.S. Application Data
(63) Continuation of application No. PCT/KR2011/001479, filed on Mar. 4, 2011.

(30) Foreign Application Priority Data
Feb. 23, 2011 (KR) 10-2011-0015954

(51) Int. Cl. A61B 17/86

(52) U.S. Cl. 606/305

(57) ABSTRACT

An extensible pedicle screw coupling device for holding a spinal rod in the head of a pedicle screw is disclosed. The device is extensible so as to fasten to the rod stably while maintaining fixing power if there is a gap between the vertebra and the rod. The device comprises pedicle screws that are inserted into the vertebrae, and a coupler in which the pedicle screw inserted into the vertebra and a head of the pedicle screw are connected, and the rod is housed therein, and fixes the rod by screw fastening the retainer to a thread formed on the top, and an extending means that can adjust the length so as to correspond to the distance between rod and vertebra is installed on the pedicle screw and the coupler.

### CROSS REFERENCE TO RELATED APPLICATIONS

This application is a continuation of International Patent Application PCT/KR2011/001479 entitled “Extendsible Pedicle Screw Coupling Device”, filed on Mar. 4, 2011, which claims priority of Korean Application No. 10-2011-0015954, filed Feb. 23, 2011.

### BACKGROUND OF THE INVENTION

The present invention relates to an extensible pedicle screw coupling device, and more specifically to an extensible pedicle screw coupling device for holding a spinal rod in the head of a pedicle screw that can be extended so as to stably fasten to a rod while maintaining the fixing power if there is a gap between a vertebra and the rod.

### DESCRIPTION OF THE RELATED ART

The most important and basic technique of modern scoliosis surgery is a rod rotation maneuver. In this method, a pedicle screw of a pedicle screw coupling device for holding a spinal rod (hereinafter, referred to as a rod) is inserted into the left and right pedicles of the vertebrae in the curved portion of the spine which is to be straightened. After that, the spinal rod is bent according to the shape of the curved spine generally in an S shape and inserted into a coupler having a tulip shape, connected to the head of the pedicle screw. Next, the rod is fixed to the coupler by tightening with a nut from the top of the coupler and then the rod is rotated about 90 degrees to straighten the curved spine. Such a rod rotation technique is basic to modern scoliosis surgery.

Although such a rod rotation technique is a very useful method, it has the following problems. The most prominent one is that trouble could occur in the process of fixing the rod to the coupler because the depth of the individual vertebra on a sagittal plane (as seen from the side with the patient laid on the operating table) is different. The depth of the lumbar vertebrae in the distal part is deeper than that of the thoracic vertebrae in the proximal part in most cases. Therefore, unless the rod shape is well bent according to the shape of the patient's vertebrae, it is not easy to connect the coupler fixed to the vertebra and rod. In other words, in the case of a rod bent in a generally S shape according to the overall shape of the vertebrae, the distance between the vertebra and the rod is considerably farther in the distal part of instrumented area compared with other parts. Therefore, once the pedicle screw is driven into the vertebra, the coupler of the pedicle screw becomes unable to house the rod.

Especially, in the case of thoracic & lumbar double curve, a problem is apt to occur in the process of fixing the screw and the rod. In the double curve, after the rod is bent in an S shape, the distal part of the rod rises in the roof direction in the process rotating the rod 90 degrees, so it is not possible to put the rod into the coupler on the distal part of the instrumented area. Further, if it is put in by force, the pedicle screw might be pulled out from the vertebra in the process of fixing the rod to the coupler with a retainer, so a serious complication can be caused.

### SUMMARY OF THE INVENTION

Accordingly, it is an object of the present invention to provide an extensible pedicle screw coupling device for holding a spinal rod in the head of a pedicle screw that has an extending means installed on a pedicle screw and a coupler so as to stably fasten to a rod while maintaining the fixing power if there is a gap between a vertebra and the rod.

In order to achieve the above-mentioned object, there is provided an extensible pedicle screw coupling device for holding a spinal rod in the head of a pedicle screw, the extensible pedicle screw coupling device comprising: a pedicle screw having a head and a threaded stem to insert into a vertebrae; a coupler having a head seat to receive the threaded stem and support the head of the pedicle screw, and a channel configured for reception of the spinal rod; a retainer configured to fix the spinal rod received in the channel of the coupler by a thread formed on an outer circumference; and an extending means which is installed on the pedicle screw and the coupler so as to adjust the length corresponding to the distance between the spinal rod and the vertebra.

In the present invention, in the method for providing an extending means to the pedicle screw, unlike the conventional method of arranging the head monolithically on the top of the pedicle screw, the head is fastened to the pedicle screw through a fastening means such as threads, and in this process the distance between the pedicle screw and the head is adjustable.

In the present invention, another method for providing an extending means to the pedicle screw is to form a sliding portion without a thread on the top of the threaded stem of the pedicle screw.

In addition, the method for providing an extending means to the coupler is to fasten an extension cap on the top of the coupler and insert a plug in the area where the rod in the coupler is arranged and arrange the rod on the top of the plug and fix it with a retainer.

Both of the above-mentioned two methods or either one of them may be provided. The extending means installed on the pedicle screw changes the length linearly or step by step, but the extending means installed on the coupler has a constant length. Therefore, it is possible for the user to properly choose any one of the two methods as necessary.

According to the present invention, the total length of the pedicle screw can be adjusted so as to prevent the pulling-out of the pedicle screw that could occur in the case of fixing the rod by force to the pedicle screw inserted into the pedicle in the situation such as different vertebral depth in scoliosis or other spinal deformities.

In addition, the present invention is not a method confined to the scoliosis of vertebrae. In a spine with a severe osteoporosis, even if the height of vertebrae on the sagittal plane is different just a little, for example, in the case of fixing the rod with a retainer, there is a fear that the spinal screw may be pulled out from the vertebrae. Therefore, the extensible pedicle screw coupling device for holding a spinal rod in the head of a pedicle screw of the present invention can be usefully used also in the surgery of osteoporosis vertebrae.

Moreover, a big change will be brought about to the current system of pedicle screw through the present invention. Currently, the size of pedicle screw is expressed in the diameter and total length of pedicle screw. For example, an expression method such as 6 mm×45 mm is used. But through the present invention, the expression system can indicate the change range of length such as 6 mm×40 to 45 mm.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a sectional view showing an extensible pedicle screw coupling device according to a first embodiment of the present invention when the head of pedicle screw is at the highest point;

FIG. 2 is a sectional view showing the extensible pedicle screw coupling device of FIG. 1 when the head of pedicle screw is at the lowest point;

FIG. 3 is a sectional view schematically showing how the coupler moves upward and downward of the pedicle screw in an extensible pedicle screw coupling device;

FIG. 4 is a schematic view showing the extensible pedicle screw coupling device of FIG. 1 installed in the vertebrae by using a rod;

FIG. 5 and FIG. 6 are sectional views showing the extensible pedicle screw coupling device positioned at opposite ends of FIG. 4, respectively;

FIG. 7 is a sectional view and partially enlarged view showing a variant of the extensible pedicle screw coupling device according to the first embodiment of the present invention;

FIG. 8 is a perspective view of an extensible pedicle screw coupling device according to a second embodiment of the present invention;

FIG. 9 is partially broken-exploded perspective view of the extensible pedicle screw coupling device of FIG. 8;

FIG. 10 is a sectional view showing the state in which the pedicle screw is assembled to the rod at the minimum length in the extensible pedicle screw coupling device of FIG. 8;

FIG. 11 is a sectional view showing the state in which the pedicle screw is assembled to the rod at the maximum length in the extensible pedicle screw coupling device of FIG. 8;

FIG. 12 is a partially broken-exploded perspective view of an extensible pedicle screw coupling device according to a third embodiment of the present invention;

FIG. 13 is a sectional view showing the state in which the pedicle screw is assembled to the rod at the maximum length in the extensible pedicle screw coupling device of FIG. 12;

FIG. 14 is a partially enlarged view of FIG. 13;

FIG. 15 is a perspective view showing the state in which a plug and an extension cap are mounted on the pedicle screw in the extensible pedicle screw coupling device according to the third embodiment of the present invention;

FIG. 16 is a partially broken-exploded perspective view of the extensible pedicle screw coupling device of FIG. 15 in which the plug and the extension cap are mounted on the pedicle screw;

FIG. 17 is a side view showing the state in which a rod cap is installed on the pedicle screw fitted with the plug and the extension cap of FIG. 15, and

FIG. 18 is a sectional view showing the state in which the rod cap is installed on the pedicle screw fitted with the plug and the extension cap of FIG. 15.

### DETAILED DESCRIPTION OF THE INVENTION

Hereinafter, preferred embodiments of the present invention will be described in more detail through the following examples. For giving numerals to various elements in the drawings, the same elements have same numerals through all drawings although they may be shown in other views. It should be noted that in the case where it is judged that a specific description for a known function with respect to the present invention and a configuration thereof may unnecessarily obscure the gist of the present invention, it will be omitted.

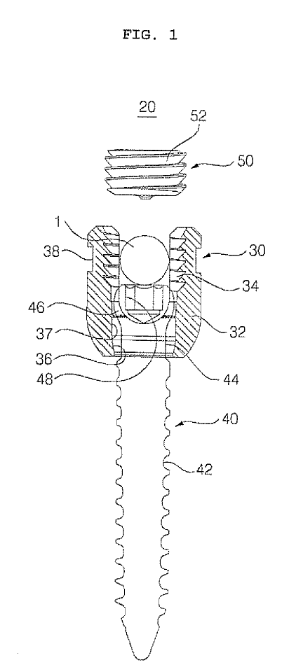

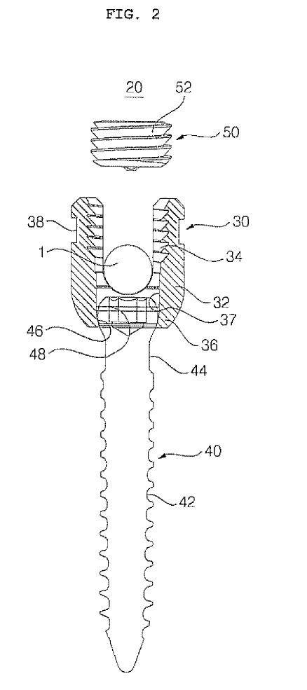

An extensible pedicle screw coupling device 20 according to a first embodiment of the present invention will be described below with reference to FIGS. 1 to 6.

First, the extensible pedicle screw coupling device 20 of this embodiment generally comprises a pedicle screw 40 having a head 46 and a threaded stem 42 to insert into a vertebrae, a coupler 30 having a head seat 36 to receive the threaded stem 42 and support the head 46 of the pedicle screw 40, and a channel configured for reception of a spinal rod (hereinafter, referred to as a rod) 1; a retainer 30 configured to fix the spinal rod 1 received in the channel of the coupler 30 by a thread 52 formed on an outer circumference; and an extending means which is provided on the pedicle screw 40 so as to be able to adjust the axial length.

In this embodiment, the screw extending means is a sliding portion 44 without a thread on the top of the pedicle screw 40.

The coupler 30 has a coupler body 32 formed in a generally U shape so as to house the rod 1 in the channel, and on the inner surface 37 of the body is formed a female thread 34 for screwing with the retainer 50. The coupler 30 has the head seat 36 formed on the bottom for the head 46 of the pedicle screw 40 to be seated therein. The head seat 36 is formed in a curved surface so as to support the bottom of the head 46. Therefore, the head 46 is pivotably installed on the head seat 36 at a predetermined angle.

The pedicle screw 40 includes the head 46 and the threaded stem 42 formed monolithically with the head 46. The head 46 has the sliding portion 44 formed on the bottom. The diameter of the sliding portion 44 is formed equal to or smaller than the minimum diameter of the head seat 36. And, the head 46 has a head wrench slot 48 formed thereon, and by inserting a tool such as a wrench (not shown) into this slot 48, it is possible to rotate the threaded stem 42 in a desired direction.

The retainer 50 can have a structure identical to the conventional art. Therefore, the retainer 50 has a male thread 52 formed on the outer circumference, and in the center of the male thread 52 is formed a retainer wrench slot for the practitioner to insert the tool to tighten or loosen the retainer 50.

Below will be described the installation process of the extensible pedicle screw coupling device 20 according to the first embodiment of the present invention. First, use a pedicle screw surgery device (not shown) which is assembled to the fastening slot 38 formed on the side of the coupler 30 to install the extensible pedicle screw coupling device 20 on a vertebrae 10. At this time, make the bottom of the coupler 30 touch or be as close as possible to the pedicle.

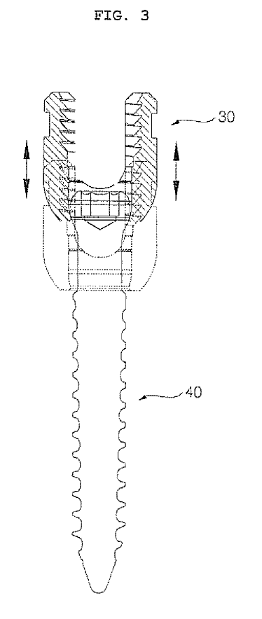

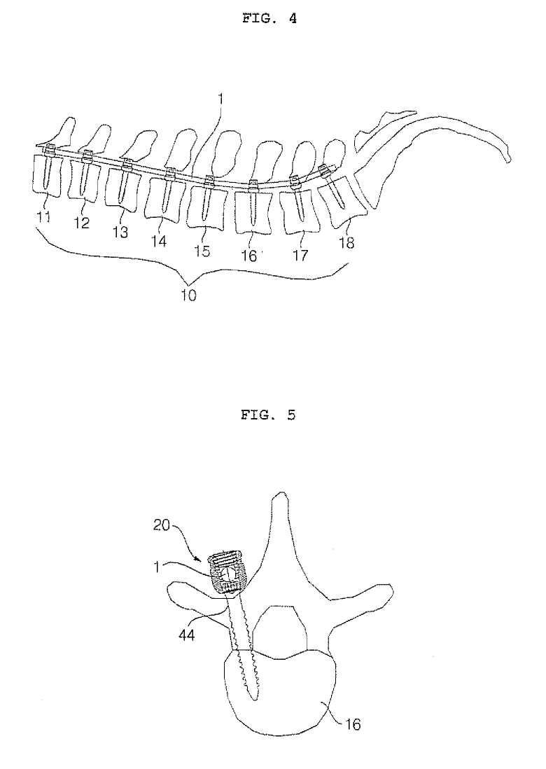

In an initial state of the pedicle screw 40, as shown in FIG. 1, the head 46 is positioned upward in the coupler 30 and the threaded stem 42 is screwed with the vertebrae 10. Therefore, in this condition, the coupler 30 can be moved upward and downward by the sliding portion 44, so vertebrae 11, 12, 13, 14, 15, 16, 17 and 18 can also be moved upward and downward of the pedicle screw 40 through the coupler 30 (See FIG. 3). Namely, in a condition like the extensible pedicle screw coupling device 20 installed in the vertebra 18 of FIG. 4, a part of the sliding portion 44 is exposed upward of the vertebra 18.

Such an up-and-down sliding motion of the coupler 30 is a major characteristic of the extensible pedicle screw coupling device 20 of this embodiment. In the conventional art, once the pedicle is screw fixed to the vertebra, additional displacement of the coupler in such a condition is impossible. However, in the extensible pedicle screw coupling device 20 of the present invention, the up-and-down sliding motion of the coupler 30 is possible through the sliding portion 44 even after the extensible pedicle screw coupling device 20 is installed in the vertebrae. As a result, the present invention has an advantage in that it is possible to properly cope with various gaps between the vertebra and the rod even with a pedicle screw of one kind and another advantage that it is possible to correct the installed condition of the pedicle screws in vertebrae.

Then, insert a separately composed rod 1 into the coupler 30. In this case, in vertebrae 11, 12, 13, 14, 15 and 16, the bottoms of the coupler 30 should touch each other, so by inserting a tool into the head wrench slot 48 and additionally rotating the pedicle screws 40, advance the pedicle screws 40 inward of vertebrae 11, 12, 13, 14, 15 and 16. And as for the vertebra 17, since the height of the bottom of the coupler 30 is lower than that of the vertebra 18 from the top of the vertebra 17 and is higher than that of vertebrae 11, 12, 13, 14, 15 and 16, adjust the advance travel by reducing the rotation rate of the pedicle screw 40. As a result, the rod 1 can be stably housed in the coupler 110.

By inserting the retainer 50 into the top side of the rod 1 and screwing the male thread 52 with the female thread 34 of the coupler 30, make the bottom of the retainer 50 press the rod 1. As the rod 1 presses the head 46, the pedicle screw 40 is fixed to the vertebra, and as shown in FIGS. 3 to 5, installation of the extensible pedicle screw coupling device 20 is completed. After that, inject filler.

It is preferable to design the length of the sliding portion 44 longer than one pitch of the threaded stem 42 of the pedicle screw 40 and shorter than the diameter of the rod 1 by considering the empirical size and spine thickness.

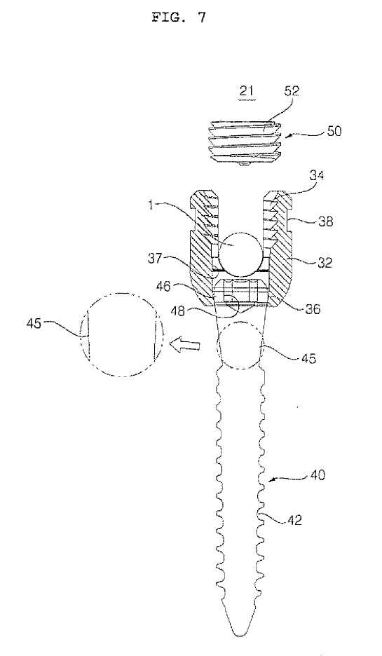

Next will be described the extensible pedicle screw coupling device 21 shown in FIG. 7. The extensible pedicle screw coupling device 21 is a variant of the extensible pedicle screw coupling device. 20 of the first embodiment. The difference between the two assemblies is that the sliding portion 45 of the extensible pedicle screw coupling device 21 is formed in a tapered type.

Namely, the sliding portion 45 is formed in such a manner that the diameter gradually increases from the pedicle screw toward the head 46, as shown in FIG. 7. Through this, as the coupler 30 rises, the head seat 36 of the coupler 30 touches the sliding portion 45. Accordingly, as the contact area between the head seat 36 and the upper side of the sliding portion 45 increases, the friction force generated between them increases, so the coupling power between the coupler 30 and the pedicle screw 40 can be improved.

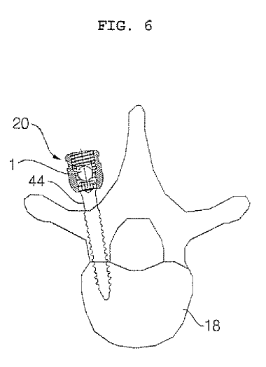

In the case of the bottom of the coupler 30 separated from the vertebra 18 as shown in FIG. 4 and FIG. 6, if the coupler 30 slides on the sliding portion 44 without resistance, the position and attitude of the coupler 30 should be maintained with another tool, etc. until the retainer 50 is screwed, when locating the rod 1 within the coupler 30 and fixing it with the retainer 50. So it can be expected a disadvantage that not only manpower is wasted but also workability decreases. However, in the extensible pedicle screw coupling device 21 of this embodiment, the coupler 30 can maintain the position and attitude by the friction force on the upper side of the sliding portion 45, so the advantage of this variant is that the convenience of the practitioner increases and unnecessary waste of time can be prevented.

In addition, the extensible pedicle screw coupling device 21 can increase the coupling power between the coupler 30 and the pedicle screw 40. Thereby, it is possible to provide an extensible pedicle screw coupling device 21 with higher reliability.

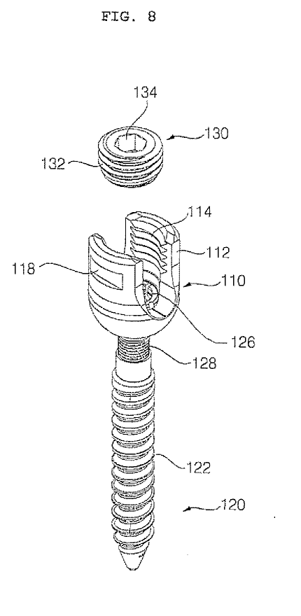

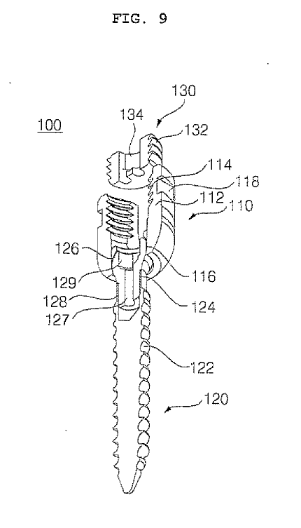

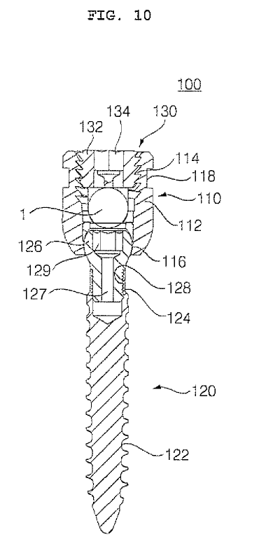

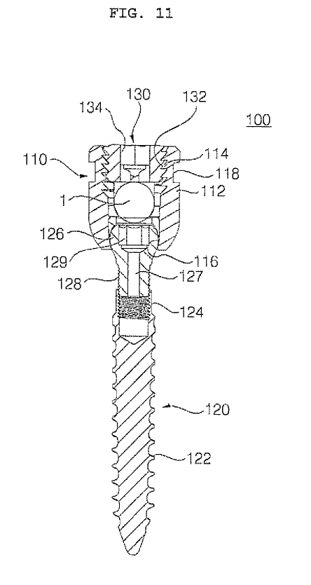

With reference to FIGS. 8 to 11, an extensible pedicle screw coupling device 100 according to a second embodiment of the present invention will be described. The extensible pedicle screw coupling device 100 includes a screw extending means installed in the pedicle screw 120.

First, the extensible pedicle screw coupling device 100 generally comprises a pedicle screw 120 which is inserted into the vertebra, and a coupler 110 in which the head 126 of the pedicle screw 120 is connected, also a rod 1 is housed in a channel of the coupler 110, and the rod 1 is fixed by screw fastening a retainer 130 to a female thread 114 formed on the upper inner wall of the head 126. The pedicle screw 120 includes a screw extending means which is installed so as to be able to adjust the axial length.

The coupler 110 has a generally U shaped coupler body 112 so as to house the rod 1 in the channel and on the inside of the body 112 is formed a female thread 114 for screwing with the retainer 130. The coupler 110 has a head seat 116 formed on the bottom for the head 126 to be seated. The head seat 116 is formed in a curved surface so as to support the bottom of the lower portion of the head 126. Accordingly, the head 126 is pivotably installed on the head seat 116 at a predetermined angle.

The pedicle screw 120 includes the head 126 and a threaded stem 122 separatably connected with the head 126 by screw fastening. The head 126 has a head male thread 128 formed on an outer circumference of a lower extended portion, and the threaded stem 122 has an insert hole into which the lower extended portion of the head is inserted and a stem female thread 124 formed on inner wall of the insert hole. In this embodiment, the screw extending means consists of the head male thread 128 of the head 126 and the stem female thread 124 of the threaded stem 122.

In the head 126 is formed a head wrench slot 129, so by inserting a tool such as wrench (not shown) into this slot and rotating the head 126, the head male thread 128 and the stem female thread 124 can be tightened or loosened. Thereby, the practitioner can adjust the gap between the head 126 and the threaded stem 122.

A head through hole 127 is formed along a center line in such a manner that it is extended from the head wrench slot 129 to the head male thread 128. Accordingly, the inside of the stem female thread 124 and the head wrench slot 129 are communicated via the head through hole 127. As a result, it is possible to firmly fix the head 126 and the threaded stem 122 since filler flows into the threaded female thread 124 via the head through hole 127 if the extensible pedicle screw coupling device 100 is installed in the vertebrae and fastened to the rod 1, and then filler, e.g. bone cement is injected therein.

The retainer 130 can have a structure identical to the conventional art. Accordingly, the retainer 130 has a male thread 132 formed on the outer circumference and the retainer wrench slot 134 in the center of the retainer 130 so that the practitioner can insert a tool into the slot to tighten or loosen the retainer 130.

Below will be described the installation process of the extensible pedicle screw coupling device 100 according to the second embodiment of the present invention. First, install the extensible pedicle screw coupling device 100 in the vertebrae using a pedicle screw surgery device (not shown). At this time, make the bottom of the coupler 110 touch or be as close as possible to the vertebra. The initial state of the pedicle screw 120 is the state in which the head 126 and the threaded stem 122 are closest.

Then, insert the individually composed rod 1 into the coupler 110. In this case, if the gap between the rod 1 and the coupler 110 is large, separate the head 126 from the threaded stem 122 by a tool inserted into the head wrench slot 129. Accordingly, the coupler 110 becomes farther from the threaded stem 122, and as a result, the rod 1 can be stably housed in the coupler 110.

Then, insert the retainer 130 into the top side of the rod 1 and screw the male thread 132 with the female thread 114 of the coupler 110 so that the bottom of the retainer 130 presses the rod 1. The rod 1 presses the head 126, so that the installation of the extensible pedicle screw coupling device 100 in the vertebrae is completed. After that, inject filler. By injecting filler, the main pedicle screw 122 and the head 126 are monolithically fixed by the filler injected via the head through hole 127 as described above.

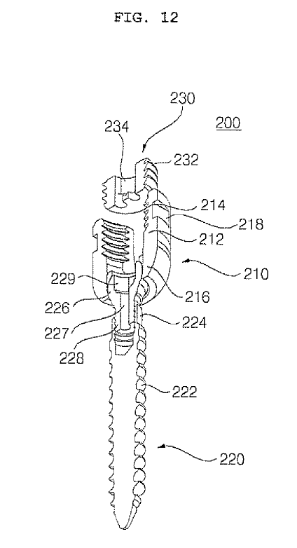

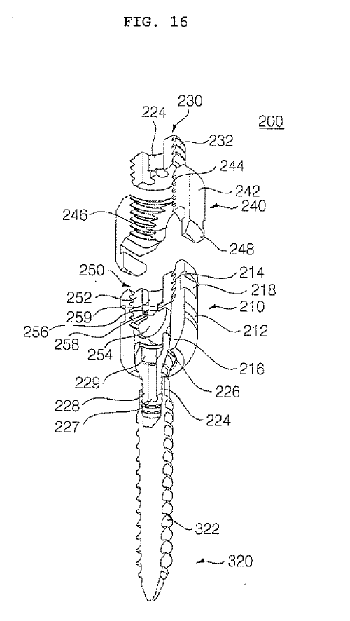

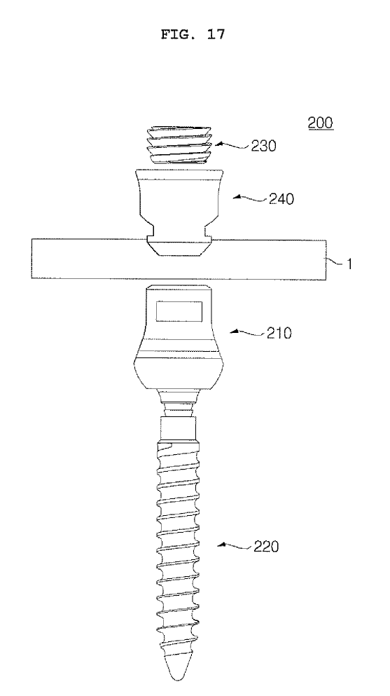

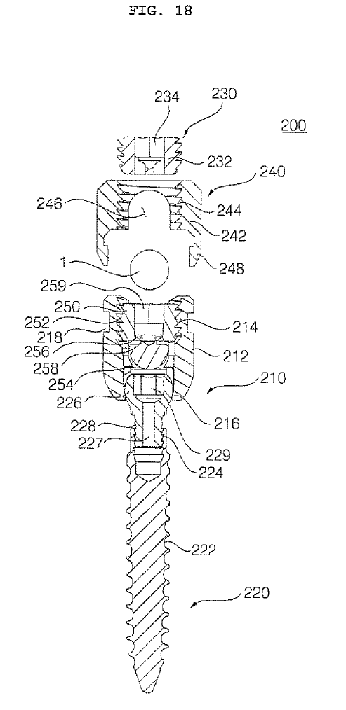

Next, with reference to FIGS. 12 to 18, an extensible pedicle screw coupling device 200 according to a third embodiment of the present invention will be described.

The extensible pedicle screw coupling device 200 generally comprises a pedicle screw 220 which is inserted into the vertebra, and a coupler 210 in which the head 226 of the pedicle screw 220 is connected, also the rod 1 is housed therein, and the rod 1 is fixed by screw fastening a retainer 230 to a coupler thread 214 formed on the upper inner wall of the head 226. The pedicle screw 220 includes a screw extending means so as to be able to adjust the length between the coupler and the pedicle screw, and a coupling-extending means is connected to the coupler 210.

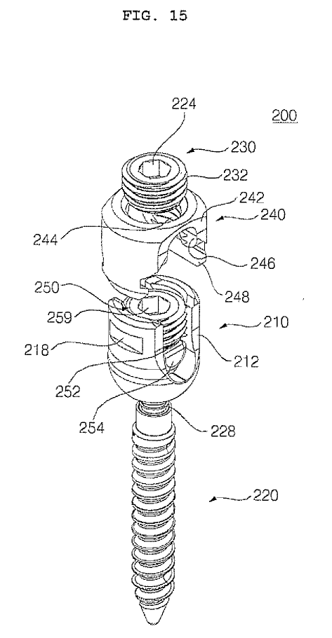

FIGS. 12 to 14 are views showing the installed state of the extensible pedicle screw coupling device 200 in the case of using only a screw extending means excluding the coupling-extending means, and FIGS. 15 to 18 are views showing the installed state in the case of using both the screw extending means and the coupling-extending means.

Although omitted here, it is possible to apply the coupling-extending means described in the third embodiment in the same way to the extensible pedicle screw coupling device 20 or 100 of the first or second embodiment. Also, it is possible to consider a variant which includes only the coupling-extending means without the screw extending means arranged in the pedicle screw 120.

The coupler 210 has a generally U shaped coupler body 212 so as to house the rod 1 therein, and on the inside is formed a coupler thread 214 for screwing with the retainer 230. The coupler 210 has a head seat 216 formed on the bottom for the head 226 of the pedicle screw 220 to be seated. The head seat 216 is formed in a curved surface so as to support the bottom of the lower portion of the head 226. Accordingly, the head 226 is pivotably installed on the head seat 216 at a predetermined angle.

On the outer circumference of the body 212 of the coupler 210 is formed a fastening slot 218. The fastening slot 218 is a component for fixing the coupler 210 when mounting the extensible pedicle screw coupling device 200 on a pedicle screw surgery device (not shown). But in the present invention, it functions as a fastening means for fixing with an extension cap 240 to be described below. And it is also possible to form in the fastening slot 218 a concave auxiliary slot again in the insert hole illustrated in FIG. 15 and form a protrusion corresponding to the auxiliary slot on the hook of the extension cap 240, to prevent coming off easily after fastening to the extension cap 240.

The pedicle screw 220 includes a head 226 and a threaded stem 222 connected with the head 226 by sawteeth coupling. The head 226 has head male sawteeth 228 formed on an outer circumference of a lower extended portion, and the threaded stem 222 has an insert hole into which the lower extended portion of the head is inserted and stem female sawteeth 224 formed on an inner wall of the insert hole corresponding to the head male sawteeth 228. In this embodiment, the screw extending means consists of head male sawteeth 228 of the head 226 and stem female sawteeth 224 of the threaded stem 222.

The head male sawteeth 228 and the stem female sawteeth 224 are, as shown in FIGS. 12 to 18, those in which round rings with right-triangle cross sections are arranged at predetermined intervals along the outer circumference. Accordingly, if these sawteeth 228 and 224 are pushed by applying force they can go in one direction but cannot go in the other direction, so arbitrary separation of the head and threaded stem is prevented. In the third embodiment of the present invention, the cross-section shape of the sawtooth is formed in such a manner that the head male sawtooth 228 can be displaced only in the direction of pushing in the stem female sawtooth 224. Thus, initially the head male sawtooth 228 is maximally separated from the stem female sawtooth 224, that is, the coupler 210 is farthest from the pedicle screw 220.

And on the head 226 is formed a head wrench slot 229, so that a tool such as a wrench (not shown) may be inserted into this slot 229 and the head 226 is rotated to fasten or loosen the head male sawtooth 226 and stem female sawtooth 224. Thereby, the practitioner can adjust the interval between the head 226 and the threaded stem 222.

A head through hole 227 is formed along center line in such a manner that it is extended from the head wrench slot 229 to the head male sawtooth 228. Accordingly, the head through hole 227 can communicate the inside of the stem female sawtooth 224 and the head wrench slot 229. As a result, it is possible to firmly fix the head 226 and the threaded stem 222 since filler flows into the threaded female thread 224 via the head through hole 227 if the extensible pedicle screw coupling device 200 is installed in the vertebrae and fastened to the rod 1, and then filler, e.g. bone cement is injected therein.

The coupling-extending means installed on the coupler 210 includes a plug 250 which is installed inside the coupler 220 to press the head of the pedicle screw 220 and an extension cap 240 that extends the length of the coupler 210 by coupling with the coupler 210.

The extension cap 240 includes a hook 248 which is fixed to the fastening slot 218 formed on the outer circumference of the coupler 220, and a cap thread 244 to which the retainer 230 is fastened, and a rod seat portion 246 through which the rod 1 can pass.

The rod seat portion 246 is formed in the cap body 242 of the extension cap 240. Namely, whereas the coupler body 220 of the coupler 210 is of a generally U shape, the cap body 242 with the rod seat portion 246 has a shape of ‘∩’. Accordingly, the rod seat portion 246 can provide space where the rod 1 is arranged. On the inner wall of the extension cap 240 is formed a cap thread 244. It is preferable that the cap thread 244 and the coupler thread 214 are threads with the same leads, diameters, and pitches. The hook 248 is preferably formed in such a size that creates a tight fit so as to be fixed stably to the coupler 210.

The plug 250 includes a plug thread 252 which is screwed with the coupler thread 214 of the coupler 210, and a head-pressing lug 254 which is rotatably mounted on the bottom of the plug 250 and is in contact with the head 226. In the top center of the plug 250 is formed a plug wrench slot 259, and by inserting a tool such as wrench (not shown) into this slot 259, it is possible to install the plug 250 in the coupler 210.

At the top end of the head-pressing lug 254 is formed a hook 256 having a neck portion, and the hook 256 is engaged in the flange 258 formed on the bottom center of the plug 250. Accordingly, the head-pressing lug 254 can maintain being assembled on the top of the head 226 despite the rotation of the plug thread 252.

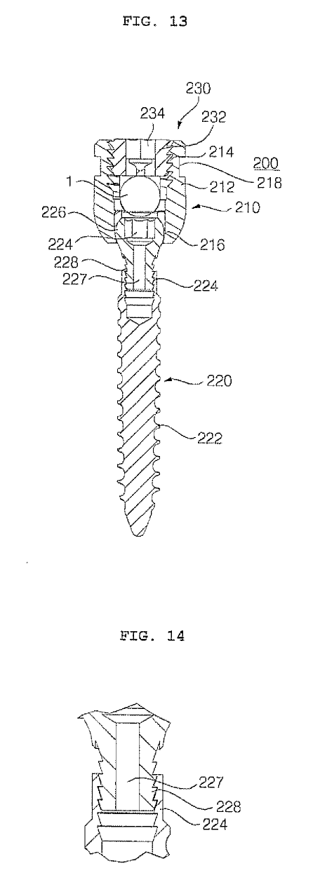

Below will be described the installation process of the extensible pedicle screw coupling device 200 of the third embodiment of the present invention. As described above, FIGS. 12 to 14 show an assembling state of the rod 1 (FIG. 13) connected to the extensible pedicle screw coupling device 200 without the extension cap 240 and plug 250, and FIGS. 15 to 18 show an assembling state of the rod (FIG. 18) connected to the extensible pedicle screw coupling device 200 using the extension cap 240 and plug 250.

First will be described how to fasten the rod 1 into the extensible pedicle screw coupling device 200 without the extension cap 240 and plug 250. First, use a pedicle screw surgery device (not shown) to install the extensible pedicle screw coupling device 200 to the vertebrae. In the initial state of the pedicle screw 220, the head 226 and the threaded stem 222 are separated maximally.

Then, insert a separately composed rod 1 into the coupler 210. In this case, if the rod 1 is closer to the vertebrae than the position where it is housed in the coupler 210, insert a tool such as wrench (not shown) into the head wrench slot 229 formed on the head 226 to make the head 226 closer to the threaded stem 222. Accordingly, the coupler 210 becomes farther from the threaded stem 222, and as a result, the rod 1 can be stably housed in the coupler 210.

Then, engage the retainer 230 in the top of the rod 1 and screw the male thread 232 into the coupler thread 214 of the coupler 210 to make the bottom of the retainer 230 press against the rod 1. As the rod 1 presses against the head 226, the pedicle screw 220 is fixed to the vertebrae, and the installation of the extensible pedicle screw coupling device is completed. After that, inject filler. When filler is injected, the main pedicle screw 222 and the head 226 are monolithically fixed by the filler that flows in via the head through hole 227 as described above.

Next will be described how the rod 1 is fastened by using the extension cap 240 and the plug 250. In this case, the length of the pedicle screw is adjusted by the principle as described above, so a description of it is omitted. The extension cap 240 and the plug 250 are used when the gap between the vertebra and the rod 1 is considerably far, so use them only when it is difficult to adjust the length by the pedicle screw 220 alone.

As described above, fasten the plug 250 in the coupler 210 with the pedicle screw 220 fixed to the vertebrae. At this time, the head-pressing lug 254 of the plug 250 is pressed to the head 226. Then, cover the extension cap 240 from above the rod 1 with the rod 1 in between so that the hook 248 of the extension cap 240 may be inserted into the fastening slot 218 of the coupler 210. Next, fix the retainer 230 to the cap thread 244 formed on the top of the extension cap 240 to complete installation of the extensible pedicle screw coupling device 200.

While the present invention has been described with reference to the preferred embodiments, it will be understood by those skilled in the related art that various modifications and variations may be made therein without departing from the scope of the present invention as defined by the appended claims.

What is claimed is:

1. An extensible pedicle screw coupling device for holding a spinal rod in the head of a pedicle screw, the extensible pedicle screw coupling device comprising: pedicle screws which are inserted into the vertebrae; and a coupler in which the head of the pedicle screw is connected, a rod is housed therein, and a retainer is screwed to a thread formed on top to fix the rod, wherein an extending means is installed on the pedicle screw and coupler so as to adjust the length corresponding to the distance between rod and vertebra.

2. The device of claim 1, wherein the extending means installed on the pedicle screw is a screw extending means for adjusting the length of the pedicle screw.

3. The device of claim 2, wherein the screw extending means includes a head which is received in the coupler and has a head male thread formed on an outer circumference of an lower extended portion; and a threaded stem which has an insert hole into which the lower extended portion of the head is inserted and a stem female thread formed on an inner wall of the insert hole to screw together with the head male thread for forming the pedicle screw, wherein the head has a head wrench slot formed thereon.

4. The device of claim 3, wherein the head has a head through hole formed therethrough to the head wrench slot, so as to communicate the insert hole of the threaded stem with the head wrench slot of the head for flowing filler injected therein.

5. The device of claim 2, wherein the screw extending means includes a head which is received in the coupler and has a plurality of head male sawteeth formed on an outer circumference of a lower extended portion; and a threaded stem which has an insert hole into which the lower extended portion of the head is inserted and a plurality of stem female sawteeth formed on an inner wall of the insert hole to screw together with the plurality of head male sawteeth for forming the pedicle screw, wherein the head has a head wrench slot formed thereon, and the stem female sawteeth and the head male sawteeth can be displaced only in the direction of reducing the length of the pedicle screw.

6. The device of claim 5, wherein the head has a head through hole formed therethrough to the head wrench slot, so as to communicate the insert hole of the threaded stem with the head wrench slot of the head for flowing filler injected therein.

7. The device of claim 1, wherein the extending means installed in the coupler is a coupling-extending means that changes the installation position of the spinal rod with respect to the coupler so as to make it farther from the pedicle screw.

8. The device of claim 7, wherein the coupling-extending means includes a plug which is installed in the coupler to press against the head of the pedicle screw; and an extension cap which has a hook fixed in the fastening slot formed on the outer circumference of the coupler, a cap thread to which the retainer is fastened, and a rod seat part for the spinal rod to pass through.

9. The device of claim 8, wherein the plug has a plug thread which is screwed with the thread of the coupler, and a head-pressing lug which is fixed rotatably to the bottom of the plug thread and is in contact with the head of the pedicle screw.

10. The device of claim 1, wherein the screw extending means is a sliding portion formed on the outer circumference of the pedicle screw near the head to adjust the distance between the spinal rod and the vertebra, wherein the head has a head wrench slot formed thereon, and the sliding portion has no thread.

11. The device of claim 10, wherein the length of the sliding portion is greater than one pitch of the thread stem of the pedicle screw and smaller than the diameter of the spinal rod.

12. The device of claim 10, wherein the sliding portion is tapered in such a manner that the diameter increases from the threaded stem toward the head, so that when the head touches the head seat of the coupler, the coupler and the head fit tightly.
